(19) United States
(12) Patent Application Publication
(10) Pub. No.: US 2025/0247599 A1
(43) Pub. Date: Jul. 31, 2025

(54) SIGNAL PROCESSING BOARD TO EXPAND FUNCTIONALITY FOR MEDICAL IMAGING SYSTEMS AND DEVICES

(71) Applicant: Boston Scientific Scimed, Inc., Maple Grove, MN (US)

(72) Inventors: Louis J. BARBATO, Franklin, MA (US); William J. ABNER, Rutland, MA (US); Kirsten VIERING, Newton, MA (US)

(73) Assignee: Boston Scientific Scimed, Inc., Maple Grove, MN (US)

(21) Appl. No.: 19/036,405

(22) Filed: Jan. 24, 2025

Related U.S. Application Data
(60) Provisional application No. 63/626,553, filed on Jan. 30, 2024.

(51) Int. Cl. H04N 23/60; A61B 1/00; A61B 1/045

(52) U.S. Cl. CPC H04N 23/60; A61B 1/00006; A61B 1/00009; A61B 1/045

(57) ABSTRACT

A computing system includes a plurality of signal processing boards configured to perform operations. Operations performed by a first signal processing board of the signal processing boards include: receiving a data signal from an image sensor of a medical device removably connected to the computing system; determining whether to route the data signal to a second or third signal processing board of the signal processing boards based on information associated with the image sensor, including characteristic(s) associated with the data signal, indicative of native or non-native support by the computing system; and, based on a determination of non-native support, providing the data signal to the third signal processing board. The third signal processing board is an expansion board removably connected to the computing system, and configured to perform operations, including: manipulating the data signal; and providing the manipulated data signal to the second signal processing board for processing.

### CROSS-REFERENCE TO RELATED APPLICATIONS

This application claims the benefit of priority to U.S. Provisional Application No. 63/626,553, filed on Jan. 30, 2024, which is incorporated by reference herein in its entirety.

### TECHNICAL FIELD

Various embodiments of the present disclosure relate generally to systems and methods for processing signals transmitted by medical devices and, more particularly, to a controller with a plurality of processing boards, including a removably connected expansion board providing functionalities for processing signals not natively supported by the controller, and related methods for routing signals to appropriate boards for processing.

### BACKGROUND

A medical device with imaging capabilities, such as an endoscope or other similar device, may include one or more image sensors configured to capture raw images of target areas within a body lumen, for example. Once raw images are captured, the medical device may be configured to transmit corresponding data signals to a controller, to which the medical device is connected, for processing. Different types or models of medical devices may contain different types of image sensors (e.g., manufactured by different imaging sensor manufacturers). Additionally, certain types of medical devices that include multiple image sensors may include different types of image sensors.

### SUMMARY OF THE DISCLOSURE

According to some aspects, the techniques described herein relate to a computing system. An example computing system includes a plurality of signal processing boards configured to perform operations, the operations including: receiving, by a first signal processing board of the plurality of signal processing boards, a data signal from an image sensor of a medical device removably connected to the computing system; determining, by the first signal processing board, whether to route the data signal to a second signal processing board or a third signal processing board of the plurality of signal processing boards based on information associated with the image sensor, including one or more characteristics associated with the data signal, indicative of native or non-native support by the computing system; based on a determination of non-native support, providing, by the first signal processing board, the data signal to the third signal processing board, wherein the third signal processing board is an expansion board removably connected to the computing system; manipulating, by the third signal processing board, the data signal; and providing, by the third signal processing board, the manipulated data signal to the second signal processing board for processing.

In any of the example computing systems described herein, the information associated with the image sensor includes whether the data signal has a first data format or a second data format. When the data signal has the first data format, the data signal is provided by the first signal processing board to the third signal processing board, and manipulating the data signal includes converting the data signal from the first data format to the second data format. In some examples, the information associated with the image sensor includes a data signal type of the data signal, and one or more communication methods or one or more communication protocols used by the image sensor to transmit the data signal.

In some aspects, the second signal processing board includes a low speed differential input and a high speed differential input, and manipulating the data signal includes routing the data signal to a particular one of the low speed differential input or the high speed differential input of the second signal processing board. In other aspects, manipulating the data signal includes modifying or altering the data signal to generate the manipulated data signal. Modifying or altering the data signal to generate the manipulated data signal includes at least one of: converting the data signal from a first data format to a second data format; converting the data signal from a first signal type to a second signal type; adjusting a length of the data signal; or adjusting a strength of the data signal. In some examples, the operations further include determining, by the first signal processing board, one or more functionalities for the third signal processing board to implement for manipulating the data signal.

In other aspects, the information associated with the image sensor is received from a memory of the medical device in response to a connection of the medical device to the computing system.

In further aspects, the data signal is a first data signal, a transmitter is associated with the medical device, and the operations further include: receiving, by the first signal processing board, a second data signal from the transmitter; determining, by the first signal processing board, to transmit the second data signal to the third signal processing board based on one or more characteristics associated with the second data signal; manipulating, by the third signal processing board, the second data signal; and providing, by the third signal processing board, the manipulated second data signal to the second signal processing board for processing. In some examples, the third signal processing board is configured to enable bidirectional data transfer between the transmitter and the computing system.

In some aspects, manipulating the data signal includes performing one or more of a plurality of manipulation functions, and the third signal processing board further includes switching circuitry configured to facilitate performance of a combination of the plurality of manipulation functions. In some examples, the third signal processing board further includes authentication circuitry configured to facilitate a determination, by the first signal processing board, of an authenticity of the third signal processing board for use in the computing system upon connection of the third signal processing board to the computing system. In other examples, the third signal processing board further includes one or more lighting elements configured to indicate a connection status of the third signal processing board to the computing system.

In other aspects, the data signal is a first data signal, the image sensor is a first image sensor, and the operations further include: receiving, by the first signal processing board, a second data signal from a second image sensor different than the first image sensor; determining, by the first signal processing board, whether to route the second data signal to the second signal processing board or the third signal processing board based on information associated with the second image sensor, including one or more characteristics associated with the second data signal, indicative of native or non-native support by the computing system; and based on a determination of native support, providing, by the first signal processing board, the second data signal to the second signal processing board for processing.

According to other aspects, the techniques described herein relate to a controller. An example controller includes an interface board, a main board, and an expansion board removably connected to the controller. The interface board is configured to: in response to a connection of a medical device to the controller, receive information associated with an image sensor of the medical device, including one or more characteristics associated with data signals generated and transmitted by the image sensor; based on the information, classify the data signals as not supported by the main board; and using the classification, route an incoming data signal received from the image sensor to the expansion board; and wherein the expansion board is configured to: manipulate the incoming data signal to generate a manipulated data signal; and provide the manipulated data signal to the main board for processing.

In any of the example controllers described herein, the information associated with the image sensor further includes information identifying hardware of the image sensor, and the one or more characteristics associated with the data signals include one or more of a data format of the data signals, a signal type of the data signals, or one or more communication methods or protocols for transmitting the data signals. In some aspects, to manipulate the incoming data signal, the expansion board is configured to one or more of: route the incoming data signal to a particular input of the main board; convert the incoming data signal from a first data format to a second data format; convert the incoming data signal from a first signal type to a second signal type; adjust a length of the incoming data signal; or adjust a strength of the incoming data signal.

According to further aspects, the techniques described herein relate to a method performed by a controller. An example method including: receiving, by a first signal processing board of the controller, information associated with an image sensor of a medical device removably connected to the controller, including one or more characteristics associated with data signals generated and transmitted by the image sensor; classifying, by the first signal processing board, the data signals as natively supported or non-natively supported by the controller; and determining, by the first signal processing board, whether to route an incoming data signal received from the image sensor to a second signal processing board or a third signal processing board of the controller based on the classification; wherein, when the data signals are classified as natively supported by the controller, providing, by the first signal processing board, the incoming data signal to the second signal processing board for processing; and wherein, when the data signals are classified as non-natively supported by the controller: providing, by the first signal processing board, the incoming data signal to the third signal processing board, wherein the third signal processing board is an expansion board removably connected to the controller; manipulating, by the third signal processing board, the incoming data signal; and providing, by the third signal processing board, the manipulated incoming data signal to the second signal processing board for processing.

In any of the example methods described herein, manipulating the incoming data signal includes one or more of: routing the incoming data signal to a particular input of the second signal processing board; converting the incoming data signal from a first data format to a second data format; converting the incoming data signal from a first signal type to a second signal type; adjusting a length of the incoming data signal; or adjusting a strength of the incoming data signal.

It is to be understood that both the foregoing general description and the following detailed description are exemplary and explanatory only and are not restrictive of the disclosed embodiments, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an example environment in which a medical imaging system including an expansion board for signal processing may be implemented, according to one or more aspects.

FIG. 2 depicts a block diagram of an example expansion board depicted in FIG. 1, according to one or more aspects.

FIG. 3 depicts an example method for routing data signals, according to one or more aspects.

FIG. 4 depicts example pathways for routing data signals, according to one or more aspects.

FIG. 5 depicts an example of a computer, according to one or more aspects.

### DETAILED DESCRIPTION

As briefly mentioned above, a medical device with imaging capabilities, such as an endoscope or other similar device, may include one or more image sensors configured to capture raw images of target areas within a body lumen during a medical procedure. Once raw images are captured, the medical device may be configured to transmit corresponding data signals to a controller, to which the medical device is connected, for processing. Different types or models of medical devices may contain different types of image sensors (e.g., manufactured by different imaging sensor manufacturers). Additionally, certain types of medical devices that include multiple image sensors may include different types of image sensors.

Image sensor manufacturers are increasingly developing unique, and in some cases, proprietary, signaling schemes for transmitting data signals from their image sensors to a receiver, such as a component of the controller. The continuous development of new signaling schemes creates unpredictability when planning for what type of electronic hardware or design to implement for the controller to support processing of data signals from a wide variety of currently available and future (e.g., not yet developed) image sensors.

Additionally, to support multiple different image sensors of one or more medical devices connected to the controller simultaneously, while minimizing a number of conductors on the medical devices, complex analog and digital switching may be utilized. However, this type of complex switching may only be implementable for known technologies (e.g., known image sensors).

Resultantly, conventional controllers are often designed to include specific hardware and/or software configured to process data signals associated with one or more types of image sensors that (i) are currently available or known and (ii) are anticipated to be incorporated within medical devices that are connectable to the controllers for use during a medical procedure. Without significant modifications or re-designs, such conventional controllers may be unable to handle the processing of data signals received from future image sensors or other types of image sensors that were not previously accounted for or supported that may, for example, use different types of data signals or data signaling schemes.

Therefore, aspects of this disclosure are directed to a plug-on or expansion board that is removably connectable to a controller, and logic implemented by the controller to enable routing of data signals to the expansion board, when appropriate. For example, the routing logic may be implemented by an interface board of the controller. When a medical device is connected to the controller, information associated with an image sensor of the medical device, may be received by the interface board. The information may include one or more characteristics associated with data signals generated and transmitted by the image sensor, such as data formats, data signal types, communication methods, and/or protocols, among other information. The interface board may use this information to classify the data signals as natively or non-natively supported by the controller.

A native support classification may indicate that the data signals are capable of being processed by a native or main processor (e.g., a main board) of the controller. Therefore, when the data signals are classified as natively supported, the interface board may proceed with routing any incoming data signals from the image sensor to the main board. Alternatively, when the data signals are classified as non-natively supported, the interface board may proceed with routing any incoming data signals from the image sensor to the expansion board. The expansion board may be configured to or may be capable of processing the incoming data signals (e.g., providing expanded functionalities to the controller). The expansion board may process and/or manipulate the incoming data signals to generate manipulated data signals that, for example, are capable of being processed by the main processing board, and provide the manipulated data signals to the main board. The expansion board may be modular, having various hardware that may be added thereto, such as additional switching circuitry and/or signal processing circuitry, to facilitate the processing or manipulation of non-natively supported data signals.

Inclusion of the expansion board, along with the routing logic, may enable the controller to support a wider variety of data formats, data signal types, communication methods, and/or protocols, and thus support multiple different image sensor types of medical devices (e.g., beyond those initially anticipated for use with the controller). Additionally, given that the expansion board is removably connectable to the controller, the expansion board may be interchangeable with other expansion boards or replaced by new expansion boards to enable the controller to support different or future-developed image sensor types. In various aspects, the expansion board may be positioned in a location of the controller that may be easily accessed by field service technicians to facilitate installation and/or removal thereof, among other service-related activities.

Further, for both data signals that are classified as natively supported and data signals that are classified as non-natively supported, multiplexing may be used such that multiple data signals may be transmitted simultaneously to the controller over a single communication channel.

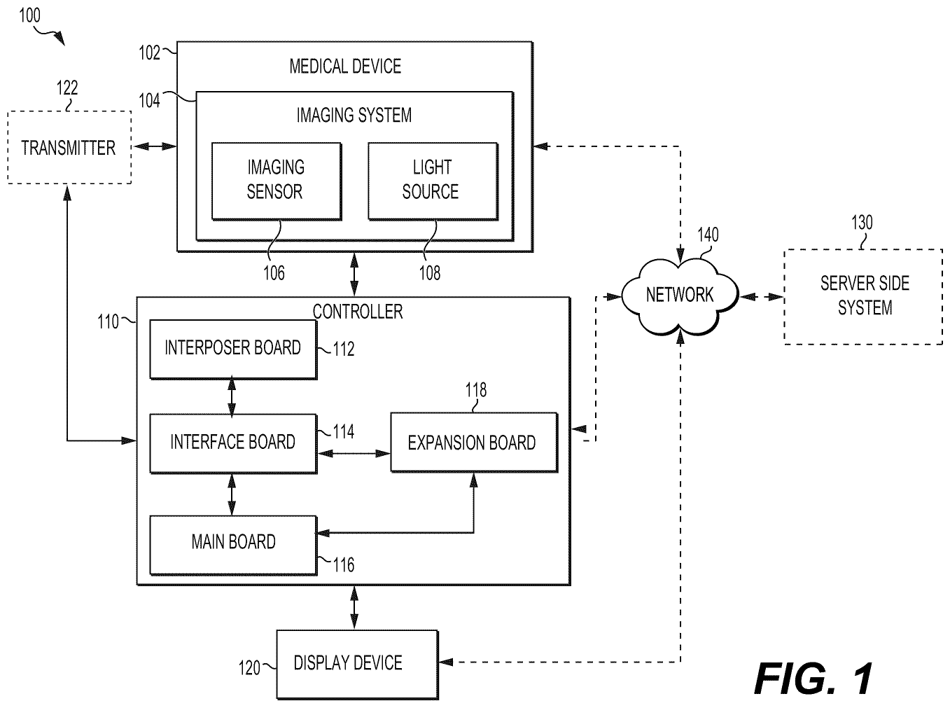

Referring now to the drawings, FIG. 1 depicts an exemplary environment 100, according to one or more aspects. Components of environment 100 may include a medical device 102, a controller 110, a display device 120, an optional transmitter 122, an optional server side system 130, and/or a network 140 over which one or more of the components may communicate with one another. While only one of each of the medical device 102, the controller 110, the display device 120, the optional transmitter 122, and the optional server side system 130 are shown in FIG. 1, the environment 100 may include multiple of one or more of the components.

The medical device 102 may be removably connected to controller 110, and used to perform a diagnostic and/or interventional medical procedure on a patient. The medical device 102 may be an endoscope, another type of scope, such as a bronchoscope, ureteroscope, duodenoscope, gastroscope, endoscopic ultrasonography (“EUS”) scope, colonoscope, laparoscope, arthroscope, cystoscope, aspiration scope, sheath, or catheter, or other similar medical device having imaging capabilities.

The medical device 102 may include an imaging system 104. The imaging system 104 may include at least one image sensor 106 and at least one light source 108. The image sensor 106 and/or the light source 108 may be located at a distal end of the medical device 102 (e.g., at a distal tip of the medical device 102). The image sensor 106 may be a particular image sensor type. For example, the image sensor 106 may include hardware associated with a manufacturer. Based on the hardware, the image sensor 106 may be configured to generate data signals of a particular data signal type (e.g., analog and/or digital). Additionally, the image sensor 106 may be configured to transmit the generated data signals using specific communication methods and/or communication protocols.

To provide an illustrative example, the image sensor 106 may be configured to capture raw images (e.g., in the form of image signals) as the distal end of the medical device 102 is inserted into and navigated through a body lumen of the patient to a target area during the medical procedure. The image sensor 106 may then transmit the image signals to the controller 110 for processing. The image sensor 106 may include one or more cameras, endoscopic viewing elements, or optical assemblies, among other similar devices. As described in more detail below, in some examples, the image sensor 106 may communicate (e.g., transmit the image signals) using a digital communication method or an analog communication method. An example digital communication method may communicate low voltage differential signaling using differential pairs. While FIG. 1 only shows the imaging system 104 of medical device 102 as having one image sensor 106, in other examples, imaging system 104 may include multiple image sensors 106, and specifically image sensors 106 of different image sensor types. Additionally or alternatively, while FIG. 1 only shows one medical device 102 connected to controller 110, in other examples, multiple medical devices 102 having the same or different types of image sensors 106 may be connected to controller 110.

The light source 108 may be configured to illuminate areas of the patient's body (e.g., the target area) during the medical procedure to facilitate imaging of the target area by the image sensor 106. The light source 108 may include one or more LEDs, incandescent light sources, optical fibers, and/or other illuminators.

The medical device 102 may be connected to the controller 110 via a wired connection. For example, the medical device 102 may be connected to the controller via an umbilicus that attaches to a connector of the medical device 102 and a connector of the controller 110. In some examples, and as mentioned above, the controller 110 may be configured to simultaneously support connection and operation of at least two medical devices 102. Among other components, the controller 110 may include one or more components configured to serve as a video processor or a signal processor (e.g., a receiver) for data signals transmitted by the medical device 102. For example, the controller 110 may include an interposer board 112, an interface board 114, a main board 116, and/or an expansion board 118.

Each of the processing boards 114, 116, and 118 may be relied upon for different functions related to the routing and/or processing of data signals transmitted by the medical device 102. The data signals may correspond to, for example, raw images or image streams (e.g., video) of a target area captured by the image sensor 106 of the medical device 102 in association with a medical procedure. The data signals may be initially received by the interposer board 112 and then routed from the interposer board 112 to the interface board 114. In some aspects, the interposer board 112 may be bypassed (or omitted in practice although shown), and the data signals may be initially received by the interface board 114.

Based on data signal classification operations performed by the interface board 114, the interface board 114 may be configured to route the data signals, via a branching or switch component of the interface board 114, to one of the main board 116 or the expansion board 118 for further processing. The main board 116 may be configured further process the data signals received directly from the interface board 114, or the processed or manipulated data signals received from the expansion board 118, to generate image data for outputting to an operator (e.g., via the display device 120).

A transmission of the data signals from the interface board 114 to the main board 116 may occur via a first routing pathway, also referred to as a “native branch”, based on the interface board 114 determining a native support classification of the data signals received from the medical device 102. The native support classification may indicate that the main board 116 of the controller 110 is configured to or capable of processing the data signals received from the medical device 102 without any hardware modifications. In some instances, software or field programmable gate array (FPGA) updates may be required, however. In some examples, the hardware of the main board 116 may be configured to support data signals of any known image sensor type incorporated within medical devices 102 that are anticipated to be connected to the controller 110 for use during a medical procedure.

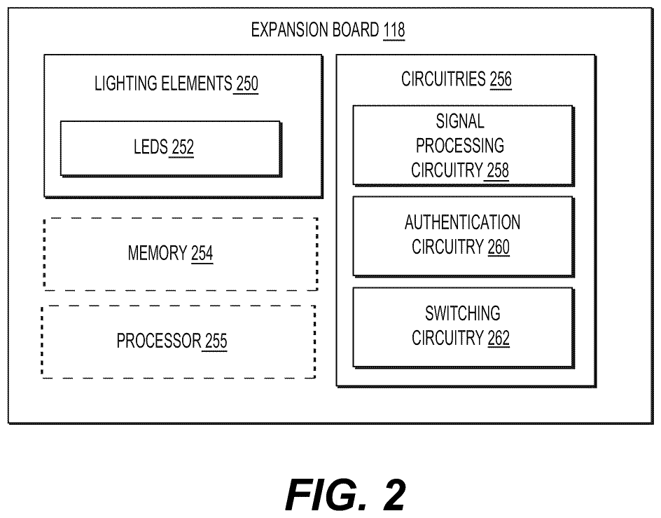

A transmission of the data signals from the interface board 114 to the expansion board 118 may occur via a second routing pathway, also referred to as a “future branch”, based on the interface board 114 determining a non-native support classification of the data signals received from the medical device 102. The non-native support classification may indicate that the hardware of main board 116 of the controller 110 is not configured to or capable of processing the data signals received from the medical device 102. However, the hardware of expansion board 118 may be configured to or may be capable of processing such data signals (e.g., providing expanded functionalities to the controller 110). In other words, the expansion board 118 may provide the hardware modifications necessary to the controller 110, so that the controller 110 may process the data signals classified as non-natively supported by, for example, routing the data signals to the expansion board 118 for manipulation and/or processing, prior to sending to the main board 116. In some examples, updates to the hardware of expansion board 118 (e.g., updates to code of the processing circuitry or FPGA) may be necessary to enable signal processing. Additionally or alternatively, software modifications and/or updates may be made to the main board 116 to support the data signal processing performed by the hardware of the expansion board 118. In other examples, when expansion board 118 includes an optional processor and memory (see FIG. 2), expansion board 118 may be configured to store software for execution, which may be updated and/or modified as needed to support the data signal processing.

Therefore, inclusion of the expansion board 118, along with the routing logic implemented by the interface board 114, may enable the controller 110 to support a wider variety of data signal types, communication methods, and/or protocols, and thus support multiple different image sensor types of medical devices (e.g., beyond those initially anticipated for use with the controller 110). Additionally, the expansion board 118 may be removably connected to one or more components within controller 110. Resultantly, the expansion board 118 may be interchangeable or replaced by new expansion boards 118 to enable controller 110 to support future technology developments. Additional description of the interface board 114, the expansion board 118, and the main board 116 are provided below.

Further, for both data signals that are classified as natively supported and data signals that are classified as non-natively supported, multiplexing may be used such that multiple data signals (digital or analog) generated by the medical device 102 may be transmitted simultaneously to the controller 110 over a single communication channel. For example, the multiple data signals may be consolidated or combined into a composite signal for transmission over the single communication channel.

The interface board 114 may be a first signal processing board of controller 110. In some examples, the interface board 114 may be a printed circuit board assembly (PCBA). The interface board 114 may include at least one memory and at least one processor. In some examples, the at least one processor may be an FPGA configured to execute various instructions stored in the at least one memory, including instructions associated with the routing logic of the branching or switching component of the interface board 114. Using this logic, the interface board 114 may be configured to classify the data signals received from the medical device 102 as either natively supported or non-natively supported. The classification may be based, at least in part on, information received from the medical device 102 upon connection of the medical device 102 to the controller 110. For example, the at least one processor of the interface board 114 may be configured to receive information (e.g., stored within a memory of the medical device 102), including at least information associated with the image sensor 106 of the medical device 102.

The information associated with the image sensor 106 may include information identifying hardware of the image sensor 106, such as a manufacturer, model number, or other similar identifier to indicate an image sensor type. In some examples, the identifier may further indicate an associated chip (e.g., a bridge chip) produced by the manufacturer for use in conjunction with the image sensor to facilitate a decoding of (e.g., a format conversion of) the signal generated by the image sensor 106, as described in more detail below. The information associated with the image sensor 106 may also include information identifying the characteristics of the data signals received from the image sensor 106, such as data signal types and formats generated, as well as communication methods and/or communication protocols for transmitting the generated data signals. Example data signal types may include an analog signal and/or a digital signal. Example data formats may include proprietary formats or standards formats for image and/or video data. Example communication protocols may include analog communication protocols (e.g., for transmission of analog signals) or digital communication protocols (e.g., for transmission of digital signals). One example digital communication protocol may include a low voltage differential signaling (LVDS) communication protocol that uses differential pairs. The differential pairs may be used in different configurations to facilitate the connection of the controller 110 to different types of medical devices 102, including medical devices 102 with single or multiple image sensors 106. The LVDS communication protocol may include, for example, positive emitter-coupled logic (PECL) or scalable low voltage signaling (SLVS). Other example communication protocols may include mobile industry processor interface (MIPI) communication protocols, among other known or future communication protocols.

If, based on the information received, the interface board 114 classifies the data signals received from the image sensor 106 of medical device 102 as natively supported by the controller 110, the interface board 114 may route the data signals to the main board 116 for further processing. If, based on the information received, the interface board 114 classifies the data signals received from the medical device 102 as non-natively supported by the controller 110, the interface board 114 may route the data signals to the expansion board 118 for further processing and manipulation.

Additionally, in some examples, the interface board 114 may be configured to determine one or more functionalities to be performed by the expansion board 118 (e.g., a type of manipulation to be performed by the expansion board 118).

The main board 116 may be a second signal processing board of the controller 110. In some examples, the main board 116 may be a PCBA. The main board 116 may include at least one memory and at least one processor. In some examples, the at least one processor may be an FPGA configured to execute various instructions stored in the at least one memory, including instructions associated with image processing. In some aspects, the at least one processor of the main board 116 may include one or more inputs, such as a low speed differential input and a high speed differential input. The low speed differential input may support processing of data signals that do not require a high data throughput, high data transmission speed, and/or high data bandwidth. The high speed differential input may support processing of data signals that require a high data throughput, high data transmission speed, and/or high data bandwidth. For example, the low speed differential input may be limited to 1.2 Gigabits per second (Gbps) per data lane. Therefore, any data signal processing requiring higher than 1.2 Gbps per data lane may be supported by the high speed differential input. An upper limit of the high speed differential input may be variable (e.g., dependent on a transceiver port of the FPGA of the main board 116). As one non-limiting example, the upper limit may be greater than 6 Gbps.

When data signals generated and transmitted by the image sensor 106 of the medical device 102 are classified as natively supported by the controller 110, the main board 116 may be configured to directly receive, from the interface board 114, any incoming data signals transmitted by the image sensor 106. Alternatively, when data signals generated and transmitted by the image sensor 106 of the medical device 102 are classified as non-natively supported by the controller 110, the main board 116 may be configured to receive manipulated data signals from the expansion board 118, as discussed in detail below. The main board 116 may be configured to further process the data signals and/or manipulated data signals to generate image or video data to provide for display to an operator of the medical device 102 (e.g., via the display device 120).

The expansion board 118 may be a third signal processing board of the controller 110. In some examples, the expansion board 118 may be a PCBA. As described in more detail with reference to FIG. 2, the expansion board 118 may include at least processing circuitry, including an FPGA, configured to process or manipulate non-natively supported data signals. In some examples, the expansion board 118 may include one or more components or sub-assemblies (e.g., third party components) that are manufactured by a separate entity than a manufacturer of the expansion board 118 and/or the controller 110. One example third party component may include a chip (e.g., a bridge chip) produced by a manufacturer of the image senor 106 for use in conjunction with the image sensor 106 to facilitate a decoding of (e.g., a format conversion of) the signal generated by the image sensor 106, as described in detail below.

When data signals generated and transmitted by the image sensor 106 of the medical device 102 are classified as non-natively supported by the controller 110, the expansion board 118 may be configured to receive, from the interface board 114, any incoming data signals transmitted by the image sensor 106. For example, the expansion board 118 may receive the data signals via the second routing pathway or future branch.

The expansion board 118 may be removably connected to the controller 110. For example, the expansion board 118 may be interchangeable or replaceable. Additionally, the expansion board 118 may be a modular processing board that may be capable of supporting various hardware updates or additions that may enable the controller 110 to process a variety of different types of data signals that may be classified as not natively supported. For example, the expansion board 118 may be configured to manipulate the data signals received from the interface board 114, and route the manipulated data signals to the main board 116 for processing, as described above.

In some examples, the manipulated data signals may be routed to either the low speed differential input or the high speed differential input of the main board 116. In other examples, the expansion board 118 may route the manipulated data signals to both the low speed differential input and the high speed differential input of the main board 116. In some aspects, the manipulation of the data signals performed by the expansion board 118 may simply be the routing of the data signals received from the medical device 102 to the low speed differential input and/or the high speed differential input. In other aspects, the manipulation may further include a modification to or alteration of the original data signals received from the medical device 102. A more detailed description of the expansion board 118 and the capabilities thereof is provided below with reference to FIG. 2.

The interface board 114, the main board 116, and the expansion board 118 have been described herein as signal processing boards (e.g., first, second, and third signal processing boards, respectively) based on each of these components being capable of processing signals. For example, each of interface board 114, the main board 116, and the expansion board 118 may be configured to perform one or more functions on a data signal. The term signal processing board is not intended to indicate any particular processing components of each of the interface board 114, the main board 116, and the expansion board 118.

The optional transmitter 122 may be a component of the medical device 102 or a component of another medical device associated with or used in conjunction with the medical device 102 to send and receive data signals (e.g., to and from controller 110). As one example, the optional transmitter 122 may be a transmitter of an ultrasound device configured to send and/or receive ultrasound signaling in a digital format. As another example, the optional transmitter 122 may be a transmitter of a sensor device configured to send and/or receive thermal, pressure, or chemical information associated with the target area. As a further example, the transmitter may be a transmitter of a device used for fluorescence lifetime capture and/or real-time navigation technologies that is configured to send and/or receive general purpose clocks. Another example may include where the optional transmitter 122 is a transmitter of a treatment device that may be configured to send and/or receive signals that have therapeutic indications, including high frequency signals (e.g., 300-500 kHz) or signals intended to drive therapeutic applications, such as lasers or similar applications. These signals may be used to perform a therapeutic function such as tissue ablation, protein denaturation, and cauterization, among others.

In some examples, the optional transmitter 122 may be coupled to or integrated with the medical device 102. For example, the optional transmitter 122 may be located in one or more working channels at a distal end of the medical device 102. In other examples, the optional transmitter 122 may be a separate device delivered to the target area (e.g., positioned internally within the patient) by the medical device 102 or other means, for example. In further examples, the optional transmitter 122 may be a separate device positioned external to the patient.

The optional transmitter 122 may be configured to send data signals to the controller 110 for processing. Additionally or alternatively, the optional transmitter 122 may be configured to receive data signals from the controller 110. For example, the expansion board 118 of the controller 110 may have expanded capabilities to act as a transmitter, in addition to a receiver of data signals. In some examples, the controller 110 may be configured to receive and process data signals transmitted by the optional transmitter 122. For example, the data signals received from the optional transmitter 122 may be routed to the interposer board 112, from the interposer board 112 to the interface board 114, and from the interface board 114 to the expansion board 118 for further processing. As described above, the interposer board 112 may be bypassed in some examples. The expansion board 118 may include the hardware (and optionally software) necessary to manipulate the data signals of the optional transmitter 122 and transmit the manipulated data signals to the main board 116 for output via the display device 120. Additionally or alternatively, the controller 110 may be configured to generate and send data signals to the optional transmitter 122. For example, the expansion board 118 of the controller 110 may include one or more capabilities or functionalities of a transmitter device, and the optional transmitter 122 may include one or more capabilities or functionalities of a receiver device. Thus, the expansion board 118 may enable bidirectional data flow or transfer between the optional transmitter 122 and the controller 110.

The display device 120 may be configured to display data associated with one or more of the medical device 102, the controller 110, and/or the optional transmitter 122. For example, displayed data may include processed images and/or information. Display of the processed images and/or information may help an operator of the medical device 102 visually navigate to and/or more clearly identify features of interest within the target area associated with the medical procedure. The display device 120 may include one or more a combination of monitors, computing device screens, touch screen display devices, etc. In some examples, the display device 120 may be a separate device from the controller 110 that is communicatively coupleable to the controller 110 via wired and/or wireless connections. In other examples, the display device 120 may be a display or screen of the controller 110 itself.

The optional server side system 130 may include one or more of remote image processing systems configured to perform at least a portion of the image processing (e.g., to conserve local resources of the controller 110, and specifically the main board 116 of the controller 110, when network connectivity is available over the network 140). Additionally or alternatively, the optional server side system 130 may include data storage systems for storing the image data generated by the controller 110. In some examples, at least one of the data storage systems may include a picture archiving and communication system (PACS) that stores the image data along with other types of imaging data from various imaging modalities (e.g., ultrasound, magnetic resonance, nuclear medicine imaging, positron emission tomography, computed tomography, mammograms, digital radiography, histopathology, etc.). Further, the optional server side system 130 may include endoscopic report writer systems configured to facilitate generation of a report based on the image data.

One or more components of the environment 100 may communicate with one another over a wired network or a wireless network, such as network 140. The network 140 may be an electronic network. The network 140 may include a wide area network (“WAN”), a local area network (“LAN”), personal area network (“PAN”), a cellular network (e.g., a 3G network, a 4G network, a 5G network, etc.), or the like. In other examples, one or more components of environment 100 may communicate and/or connect to the network over universal serial bus (USB) or other similar local, low latency connections or direct wireless protocol. The components of environment 100 may be connected via the network, using one or more standard communication protocols, such that the component may transmit and receive communications from each other across the network.

Although various components in the environment 100 are depicted as separate components in FIG. 1, it should be understood that a component or portion of a component in environment 100 may, in some embodiments, be integrated with or incorporated into one or more other components. For example, the optional transmitter 122 may be integrated with the medical device 102, and/or the display device 120 may be integrated with the controller 110. In some embodiments, operations or aspects of one or more of the components discussed above may be distributed amongst one or more other components. Any suitable arrangement and/or integration of the various systems and devices of the environment 100 may be used.

It should be understood that techniques according to this disclosure may be adapted to any medical imaging system that includes a controller capable of supporting connections to and operations of different types of medical devices having varying image sensor types included therein. It should also be understood that the examples above are illustrative only. The techniques and technologies of this disclosure may be adapted to any suitable activity.

FIG. 2 depicts a block diagram of the expansion board 118, according to one or more aspects. The expansion board 118 may include one or more lighting elements 250, an optional memory 254, an optional processor 255, and a plurality of circuitries 256. The lighting elements 250 may include one or more light emitting diodes (LEDs) 252. The circuitries 256 may include signal processing circuitry 258, an authentication circuitry 260, and/or a switching circuitry 262.

The expansion board 118 may be removably connected to or removably installed in the controller 110. In some examples, the expansion board 118 may be removably installed at the interface board 114. For example, the expansion board 118 may be physically and/or electrically coupled to the interface board 114. In other examples, the expansion board 118 may be removably installed at the interposer board 112.

To support different types of image sensors 106, medical devices 102, or transmitters 122 beyond those initially anticipated for use with the controller 110 and/or to support future technology developments that are not yet known, the expansion board 118 may be replaceable and/or interchangeable with different or new expansion boards 118 including hardware and/or software configured to or capable of supporting data signal processing associated therewith. Additionally, the expansion board 118 may support hardware and/or software updates. In some examples, the hardware and/or software updates may be performed in the field (e.g., at a location of the controller 110). To facilitate field servicing, the expansion board 118 may be positioned within the controller 110 in a location that may be easily accessible by a field service technician for installation and/or removal. For example, the expansion board 118 may be removably connected to the controller 110 in the field as a hardware upgrade to the controller 110 by a field service technician (e.g., by replacing currently connected expansion board 118 with a new expansion board 118). Alternatively, the controller 110 may be replaced by a new controller 110 having the new expansion board 118 that is already removably connected or installed.

During installation, the LEDs 252 may indicate a successful physical and electrical connection of the expansion board 118 to the controller 110. For example, the LEDs 252 may emit one or more light pulses as a visual cue to confirm that the expansion board 118 has been installed correctly. Additionally, in some examples, the expansion board 118 may be designed such that the connection of the expansion board 118 to the controller 110 cannot be misaligned or inadequately connected. Further, the expansion board 118 may be designed such that the installation thereof can be performed using a single hand to facilitate field servicing.

The optional memory 254 may be or include an electrically erasable programmable read-only memory (EEPROM). The optional memory 254 may be configured to store parameters, among other instructions, for use in the data signal processing. The parameters stored may be distinct from parameters received from the memory of the medical device 102 upon connection of the medical device 102 to the controller 110, as described in detail below. Additionally or alternatively, the optional memory 254 may be configured to store information to enable authentication of the expansion board 118. The optional processor 255 may be a microcontroller, for example, configured to execute instructions for data signal processing stored in the optional memory 254. The optional processor 255 may be in communication with (e.g., to control) one or more of the circuitries 256.

The signal processing circuitry 258 may include an FPGA configured to execute operations associated with the processing or manipulating of data signals that are routed to the expansion board 118 by the interface board 114. For example, one or more of a plurality of manipulation functions may be performed to manipulate the data signals. Resultantly, the expansion board 118 may provide additional functionalities without the need to redesign the hardware of the interface board 114, the main board 116, or the controller 110, generally.

One example type of manipulation function may include a routing of the data signals to the low speed differential input and/or the high speed differential input of the main board 116. In other words, the expansion board 118 may provide straight through connections (e.g., may not modify or alter the data signals), but may route certain data signals received from the interface board 114 to the low speed and/or high speed differential inputs of the main board 116. Another example type of manipulation function that may be performed in conjunction with or independently from the above-described routing may include a modification or alteration of the data signals.

One example modification may include converting a data format of the data signals from a first data format to a second data format. The first data format may be a proprietary image and/or video format or other similar format that is not compatible with the main board 116. The second data format may be a standard image and/or video format that is compatible with (e.g., capable of being processed by) the main board 116. The expansion board 118 may include one or more chips, such as one or more bridge chips corresponding to particular types of image sensors 106, to facilitate the conversion of the proprietary image and/or video format of the image sensors 106 to standard image and/or video formats. Another example modification may include a changing of a signal type of the data signals from a first signal type to a second signal type (e.g., from an analog signal to a digital signal or vice versa). Further example modifications may include an adjustment of signal length and/or an adjustment of signal strength. For example, the expansion board 118 may be configured to add retimers or signal conditioners to data lines to adjust for electrical length or input signal strength of the data signals.

In some examples, the signal processing circuitry 258 may be hardcoded with logic or instructions to determine which type of manipulation (e.g., routing and/or modification and which type of modification) to perform as part of the data signal processing. In other examples, the determination may be performed by software and/or firmware of the interface board 114 and/or the main board 116, and the signal processing circuitry 258 may be controlled based on the determination. In further examples, when the expansion board 118 includes the optional memory 254 and the optional processor 255, the optional processor 255 may be configured to perform the determination based instructions stored in the optional memory 254.

The signal processing circuitry 258 may further be configured to control operations of the switching circuitry 262. The switching circuitry 262 may enable different combinations of one or more of the above-described manipulation functions of the expansion board 118 to be performed. For example, one switch of the switching circuitry 262 may cause LVDS data to be routed to the high speed differential input of the main board 116. The same switch may be used to route other data signals to the retimers, and then to the low speed differential input of the main board 116. Additional switches of the switching circuitry 262 may be used to facilitate routing and/or processing of data signals associated with other components of the environment 100, such as from the optional transmitter 122 or components of the medical device 102 other than the image sensor 106.

The authentication circuitry 260 may enable the controller 110 to detect whether the expansion board 118 is valid at a time of installation. For example, the one or more processors of the interface board 114 may be configured to check the authenticity of the expansion board 118 via the authentication circuitry 260 to confirm that the expansion board 118 is authentic or valid. For example, the authentication circuitry 260 may be in communication with an encrypted memory device (e.g., the optional memory 254) of the expansion board 118, and facilitate a provision of a key from the encrypted memory device to the interface board 114. The at least one processor of the interface board 114 may be able to read the key to confirm the expansion board 118 is authentic or valid.

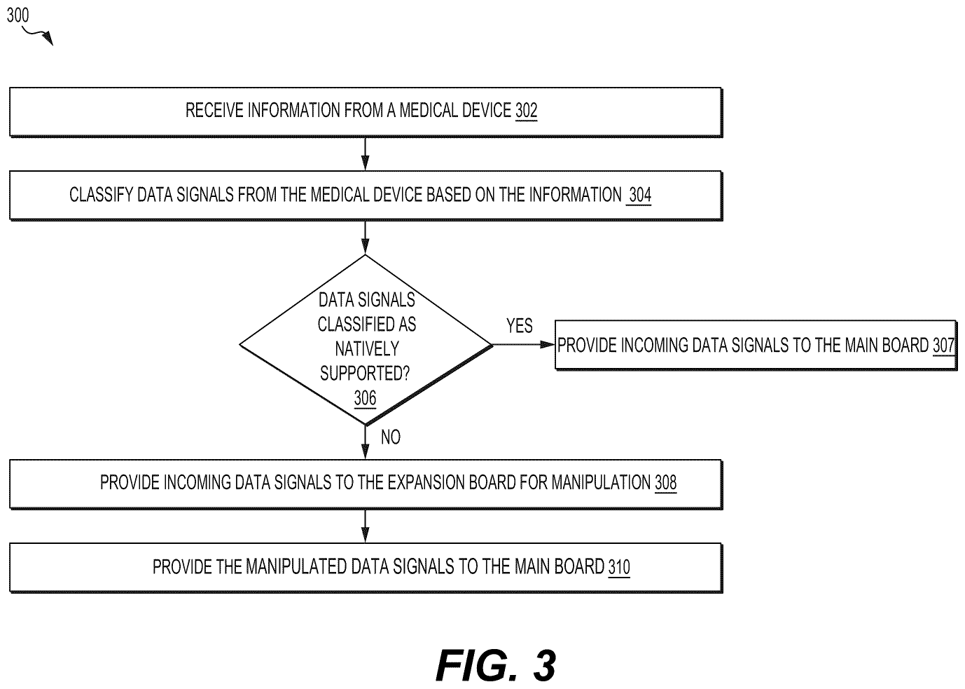

FIG. 3 depicts an exemplary method 300 for routing data signals, according to one or more aspects. In some examples, one or more steps or decisions of the method 300 may be performed by the interface board 114 of the controller 110. At step 302, the interface board 114 may be configured to receive information from the medical device 102. In some examples, the information may be received upon connection of the medical device 102 to the controller 110. For example, the information received may be included within one or more initialization signals received from the medical device 102 during an initialization stage triggered by the connection of the medical device 102 to the controller 110. The information may be stored within and accessed from the memory of the medical device 102 for transmission via the initialization signals.

The initialization signals may include information pertaining to or associated with the image sensor 106 of the medical device 102. For example, the information may include information identifying hardware of the image sensor 106, such as a manufacturer, model number, or other similar identifier to indicate an image sensor type. The information may also include information identifying the characteristics of the data signals received from the image sensor 106, such as data signal types or formats generated, as well as communication methods and/or communication protocols for transmitting the generated data signals. Example data signal types may include an analog signal and/or a digital signal. Example data formats may include proprietary formats or standards formats for image and/or video data. Example communication protocols may include analog communication protocols (e.g., for transmission of analog signals) or digital communication protocols (e.g., for transmission of digital signals). One example digital communication protocol may include a LVDS communication protocol that uses differential pairs. The differential pairs may be used in different configurations to facilitate the connection of the controller 110 to different types of medical devices 102, including medical devices 102 with single or multiple image sensors 106. The LVDS communication protocol may include, for example, PECL or SLVS. Other example communication protocols may include mobile MIPI communication protocols, among other known or future communication protocols.

In examples, where multiple medical devices 102 are connected to controller 110, information may be received from each medical device 102, such that information pertaining to the respective image sensor 106 of each medical device 102 is received. Additionally or alternatively, when any one or more medical devices 102 connected to the controller 110 include multiple image sensors 106, information pertaining to each of the image sensors 106 of the respective medical device 102 may be received as part of the information at step 302.

At step 304, the interface board 114 may classify data signals from the medical device 102, and specifically image and/or video data signals that will be generated and transmitted by the image sensor 106, based on the information received at step 302. The data signals may be classified as natively supported or non-natively supported by the controller 110. The classification may be used to determine, by the interface board 114, whether to route the data signals, upon receipt, to the main board 116 or the expansion board 118 for further processing.

A native support classification may indicate that at least the hardware of main board 116 of the controller 110 is configured to or capable of processing the data signals received from the image sensor 106 of medical device 102 without any modifications. In some instances, software or field programmable gate array (FPGA) updates may be required, however. Therefore, based on the native support classification, the interface board 114 may determine to route the data signals, upon receipt, to the main board 116.

The non-native support classification may indicate that the hardware of main board 116 of the controller 110 is not configured to or capable of processing the data signals received from the medical device 102. However, the hardware of expansion board 118 may be configured to or may be capable of processing such data signals (e.g., providing expanded functionalities to the controller 110). In other words, the expansion board 118 may provide the hardware modifications necessary to the controller 110, so that the controller 110 may process the data signals classified as non-natively supported by, for example, routing the data signals to the expansion board 118 for manipulation and/or processing, prior to sending to the main board 116.

At decision 306, the interface board 114 may determine whether the data signals are classified as natively supported based on the classification performed at step 304. The classification may indicate a routing path or branch via which data signals are to be provided, as described in detail below with respect to steps 307 and 308. In some examples, the classification and/or the routing path or branch indicated by the classification may be stored in association with the image sensor 106 such that when incoming data signals from the image sensor 106 are received, the incoming data signals may be routed based on the stored classification and/or routing path. In examples where multiple medical devices 102 are connected to controller 110 and/or where one or more medical devices 102 connected to the controller 110 include multiple image sensors 106, the classification and/or routing path indicated by the classification for each image sensor 106 may be stored in association with the image sensor 106. Such storage may facilitate routing, particularly, as an operator switches between use of medical devices 102 and/or image sensors 106 of a given medical device 102 during a medical procedure.

If, at decision 306, the data signals are classified as natively supported, the method 300 proceeds to step 307. At step 307, incoming data signals, once received from the medical device 102, are provided to the main board 116 for further processing (e.g., to generate images and/or videos for output via the display device 120). For example, the interface board 114, upon receiving the incoming data signals via the interposer board 112 or directly (if the interposer board 112 is bypassed), may route the data signals to the main board 116 based on the native support classification.

Otherwise, if at decision 306, the data signals are classified as non-natively supported, the method 300 proceeds to step 308. At step 308, incoming data signals, once received from the medical device 102, are provided to the expansion board 118 for manipulation. For example, the interface board 114, upon receiving the incoming data signals via the interposer board 112 or directly (if the interposer board 112 is bypassed), may route the incoming data signals to the expansion board 118 based on the non-native support classification. As described in detail with reference to FIG. 2, example types of manipulation performed by the expansion board 118 may include a routing of the incoming data signals to the low speed differential input and/or the high speed differential input of the main board 116 and/or a modification or alteration of the incoming data signals. Example modifications or alterations may include a converting of a data format or a signal type of the data signals, an adjustment of signal length, and/or an adjustment of signal strength.

At step 310, the expansion board 118 may be configured to provide the manipulated data signals to the main board 116 for further processing (e.g., to generate images and/or videos for output via the display device 120). In some examples, the expansion board 118 may be configured route the manipulated data signals to either the low speed and/or high speed differential inputs of the main board 116.

While the method 300 depicted in FIG. 3 describes steps and/or decisions performed by the interface board 114 for routing data signals received from the medical device 102, and more specifically the image sensor 106 thereof, the same or similar steps may be performed for data signals received from the optional transmitter 122. For example, at step 302, the interface board 114 may be configured to receive information from the optional transmitter 122, and at least one or more of the steps or decisions 304, 306, 308, and 310 may be repeated based on the data signal received from the optional transmitter 122. For example, the data signal may be classified as not natively supported, and provided to the expansion board 118 for manipulation.

Accordingly, certain aspects may include classification-based routing of data signals. The method 300 described above is provided merely as an example, and may include additional, fewer, different, or differently arranged steps and/or decisions than depicted in FIG. 3.

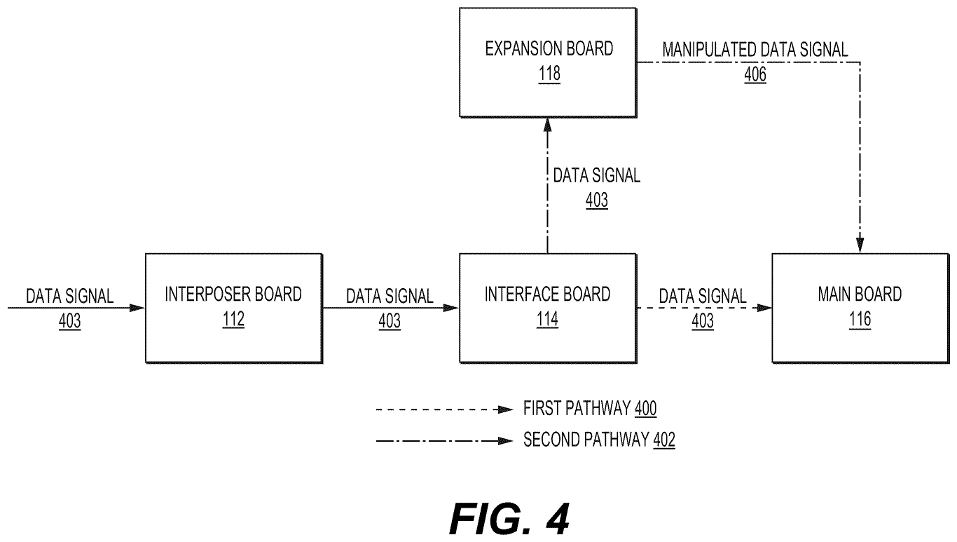

FIG. 4 depicts two exemplary pathways for routing data signals, according to one or more aspects. For example, the interface board 114 may be configured to route a data signal 403 received from the image sensor 106 of the medical device 102 along one of a first pathway 400 or a second pathway 402. A determination of whether to route the data signal 403 via the first pathway 400 or the second pathway 402 may be based on a classification performed by the interface board 114 to classify data signals generated and transmitted by the image sensor 106 (or alternatively the optional transmitter 122), such as the data signal 403, as one of natively supported or non-natively supported by the controller 110, as described in detail with reference to FIG. 3.

To provide an illustrative example, the data signal 403 may be received by the interposer board 112 from the image sensor 106 of the medical device 102 (or alternatively the optional transmitter 122). The data signal 403 may then be transmitted from the interposer board 112 to the interface board 114. In other examples, the interposer board 112 may be bypassed and the data signal 403 may be received directly by the interface board 114.

Based on a native support classification, the data signal 403 may be transmitted along the first pathway 400 to the main board 116 for further processing. Alternatively, based on a non-native support classification, the data signal 403 may be transmitted along the second pathway 402 to the expansion board 118. The expansion board 118 may be configured to manipulate the data signal 403 to generate a manipulated data signal 406, and provide the manipulated data signal 406 to the main board 116 for further processing.

The main board 116 may be configured to process the data signal 403 or the manipulated data signal 406 to generate and output processed images or video data for display to an operator of the medical device 102 via the display device 120.

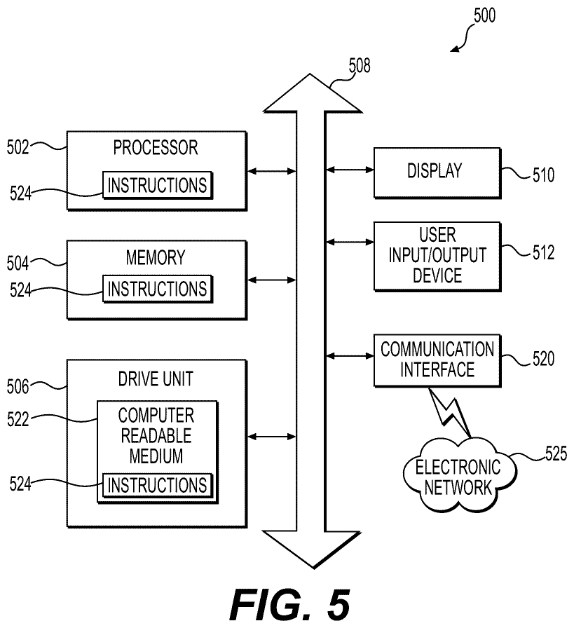

FIG. 5 depicts an example of a computer 500, according to one or more aspects. FIG. 5 is a simplified functional block diagram of computer 500 that may be configured as a device for executing processes, steps, or operations depicted in, or described with respect to FIGS. 1-4, and according to exemplary embodiments of the present disclosure. For example, the computer 500 may be configured as one or more of the medical device 102, the controller 110, the interposer board 112, the interface board 114, the expansion board 118, the main board 116, the display device 120, the optional transmitter 122, the optional server side system 130, and/or another device or component according to exemplary aspects of this disclosure. In various aspects, any of the systems herein may be or include the computer 500 including, e.g., a data communication interface 520 for packet data communication. The computer 500 may communicate with one or more other computers, for example, using an electronic network 525 (e.g., via the data communication interface 520). The electronic network 525 may include a wired or wireless network, for example, similar to the network 140 depicted in FIG. 1.

The computer 500 also may include a central processing unit (“CPU”), in the form of one or more processors 502, for executing program instructions 524. In some examples, the processors may include FPGAs. The program instructions 524 may include at instructions for performing data signal routing logic, data signal processing or manipulation, and/or image processing.

The computer 500 may include an internal communication bus 508. The computer 500 may also include a drive unit 506 (such as read-only memory (ROM), hard disk drive (HDD), solid-state disk drive (SDD), etc.) that may store data on a computer readable medium 522 (e.g., a non-transitory computer readable medium), although the computer 500 may receive programming and data via network communications. The computer 500 may also have a memory 504 (such as random-access memory (RAM)) storing the instructions 524 for executing techniques presented herein. It is noted, however, that in some aspects, the instructions 524 may be stored temporarily or permanently within other modules of the computer 500 (e.g., the processor 502 and/or the computer readable medium 522). The computer 500 also may include user input and output devices 512 and/or a display 510 to connect with input and/or output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. A computing system, comprising:
a plurality of signal processing boards configured to perform operations, the operations comprising:
receiving, by a first signal processing board of the plurality of signal processing boards, a data signal from an image sensor of a medical device removably connected to the computing system;
determining, by the first signal processing board, whether to route the data signal to a second signal processing board or a third signal processing board of the plurality of signal processing boards based on information associated with the image sensor, including one or more characteristics associated with the data signal, indicative of native or non-native support by the computing system;
based on a determination of non-native support, providing, by the first signal processing board, the data signal to the third signal processing board, wherein the third signal processing board is an expansion board removably connected to the computing system;
manipulating, by the third signal processing board, the data signal; and
providing, by the third signal processing board, the manipulated data signal to the second signal processing board for processing.

2. The computing system of claim 1, wherein the information associated with the image sensor includes whether the data signal has a first data format or a second data format.

3. The computing system of claim 2, wherein when the data signal has the first data format, the data signal is provided by the first signal processing board to the third signal processing board, and wherein manipulating the data signal includes converting the data signal from the first data format to the second data format.

4. The computing system of claim 2, wherein the information associated with the image sensor includes a data signal type of the data signal, and one or more communication methods or one or more communication protocols used by the image sensor to transmit the data signal.

5. The computing system of claim 1, wherein the second signal processing board includes a low speed differential input and a high speed differential input, and manipulating the data signal includes routing the data signal to a particular one of the low speed differential input or the high speed differential input of the second signal processing board.

6. The computing system of claim 1, wherein manipulating the data signal includes modifying or altering the data signal to generate the manipulated data signal.

7. The computing system of claim 6, wherein modifying or altering the data signal to generate the manipulated data signal comprises at least one of:
converting the data signal from a first data format to a second data format;
converting the data signal from a first signal type to a second signal type;
adjusting a length of the data signal; or
adjusting a strength of the data signal.

8. The computing system of claim 1, further comprising:
determining, by the first signal processing board, one or more functionalities for the third signal processing board to implement for manipulating the data signal.

9. The computing system of claim 1, wherein the information associated with the image sensor is received from a memory of the medical device in response to a connection of the medical device to the computing system.

10. The computing system of claim 1, wherein the data signal is a first data signal, a transmitter is associated with the medical device, and the operations further comprising:
receiving, by the first signal processing board, a second data signal from the transmitter;
determining, by the first signal processing board, to transmit the second data signal to the third signal processing board based on one or more characteristics associated with the second data signal;
manipulating, by the third signal processing board, the second data signal; and
providing, by the third signal processing board, the manipulated second data signal to the second signal processing board for processing.

11. The computing system of claim 10, wherein the third signal processing board is configured to enable bidirectional data transfer between the transmitter and the computing system.

12. The computing system of claim 1, wherein manipulating the data signal includes performing one or more of a plurality of manipulation functions, and the third signal processing board further includes switching circuitry configured to facilitate performance of a combination of the plurality of manipulation functions.

13. The computing system of claim 1, wherein the third signal processing board further includes authentication circuitry configured to facilitate a determination, by the first signal processing board, of an authenticity of the third signal processing board for use in the computing system upon connection of the third signal processing board to the computing system.

14. The computing system of claim 1, wherein the third signal processing board further includes one or more lighting elements configured to indicate a connection status of the third signal processing board to the computing system.

15. The computing system of claim 1, wherein the data signal is a first data signal, the image sensor is a first image sensor, and the operations further comprise:
receiving, by the first signal processing board, a second data signal from a second image sensor different than the first image sensor;
determining, by the first signal processing board, whether to route the second data signal to the second signal processing board or the third signal processing board based on information associated with the second image sensor, including one or more characteristics associated with the second data signal, indicative of native or non-native support by the computing system; and
based on a determination of native support, providing, by the first signal processing board, the second data signal to the second signal processing board for processing.

16. A controller, comprising:
an interface board;
a main board; and
an expansion board removably connected to the controller;
wherein the interface board is configured to:
in response to a connection of a medical device to the controller, receive information associated with an image sensor of the medical device, including one or more characteristics associated with data signals generated and transmitted by the image sensor;
based on the information, classify the data signals as not supported by the main board; and
using the classification, route an incoming data signal received from the image sensor to the expansion board; and
wherein the expansion board is configured to:
manipulate the incoming data signal to generate a manipulated data signal; and
provide the manipulated data signal to the main board for processing.

17. The controller of claim 16, wherein the information associated with the image sensor further includes information identifying hardware of the image sensor, and the one or more characteristics associated with the data signals include one or more of a data format of the data signals, a signal type of the data signals, or one or more communication methods or protocols for transmitting the data signals.

18. The controller of claim 16, wherein, to manipulate the incoming data signal, the expansion board is configured to one or more of:
route the incoming data signal to a particular input of the main board;
convert the incoming data signal from a first data format to a second data format;
convert the incoming data signal from a first signal type to a second signal type;
adjust a length of the incoming data signal; or
adjust a strength of the incoming data signal.

19. A method performed by a controller, comprising:
receiving, by a first signal processing board of the controller, information associated with an image sensor of a medical device removably connected to the controller, including one or more characteristics associated with data signals generated and transmitted by the image sensor;
classifying, by the first signal processing board, the data signals as natively supported or non-natively supported by the controller; and
determining, by the first signal processing board, whether to route an incoming data signal received from the image sensor to a second signal processing board or a third signal processing board of the controller based on the classification;
wherein, when the data signals are classified as natively supported by the controller, providing, by the first signal processing board, the incoming data signal to the second signal processing board for processing; and
wherein, when the data signals are classified as non-natively supported by the controller:
providing, by the first signal processing board, the incoming data signal to the third signal processing board, wherein the third signal processing board is an expansion board removably connected to the controller;
manipulating, by the third signal processing board, the incoming data signal; and
providing, by the third signal processing board, the manipulated incoming data signal to the second signal processing board for processing.

20. The method of claim 19, wherein manipulating the incoming data signal includes one or more of:
routing the incoming data signal to a particular input of the second signal processing board;
converting the incoming data signal from a first data format to a second data format;
converting the incoming data signal from a first signal type to a second signal type;
adjusting a length of the incoming data signal; or
adjusting a strength of the incoming data signal.
